(19) United States
(12) Patent Application Publication
(10) Pub. No.: US 2025/0271374 A1
(43) Pub. Date: Aug. 28, 2025

(54) IN-SITU VARIABLE TEMPERATURE BOW METROLOGY

(71) Applicant: Tokyo Electron Limited, Tokyo (JP)

(72) Inventors: Joshua BAILLARGEON, Albany, NY (US); Andrew METZ, Albany, NY (US); Quoc-Bao VU, Albany, NY (US)

(73) Assignee: Tokyo Electron Limited, Tokyo (JP)

(21) Appl. No.: 18/588,318

(22) Filed: Feb. 27, 2024

(51) Int. Cl. G01N 25/72; G01N 33/00

(52) U.S. Cl. CPC G01N 25/72; G01N 33/0095

(57) ABSTRACT

Aspects of the present disclosure provide a measurement device. For example, the measurement device can include a measurement vessel providing an enclosed space for a wafer to be placed therein, and a plurality of pins provided in the measurement vessel for the wafer to rest thereon. The pins can be configured to be raised to a first position and lowered to a second position. The measurement device can also include a heater configured to control the enclosed space to reach a first temperature elevated from a second temperature, and a bow measurement device configured to measure the wafer to identify a bow of the wafer when the wafer rests on the pins and the pins are raised to the first position.

### FIELD OF THE INVENTION

The present disclosure relates to integration and wafer processing, and, more particularly, to in-situ variable temperature bow metrology.

### BACKGROUND

The background description provided herein is for the purpose of generally presenting the context of the disclosure. Work of the presently named inventors, to the extent the work is described in this background section, as well as aspects of the description that may not otherwise qualify as prior art at the time of filing, are neither expressly nor impliedly admitted as prior art against the present disclosure.

In an ideal situation, a wafer is planar. However, after various processing steps are executed on the wafer, the wafer tends to have a bow and is thus non-planar. Such a non-planar wafer may be considered unstable or even be broken during subsequent processing steps at elevated temperatures.

### SUMMARY

Aspects of the present disclosure provide a measurement device. For example, the measurement device can include a measurement vessel providing an enclosed space for a wafer to be placed therein, and a plurality of pins provided in the measurement vessel for the wafer to rest thereon. The pins can be configured to be raised to a first position and lowered to a second position. The measurement device can also include a heater configured to control the enclosed space to reach a first temperature elevated from a second temperature, and a bow measurement device configured to measure the wafer to identify a bow of the wafer when the wafer rests on the pins and the pins are raised to the first position.

In an embodiment, the measurement vessel can further include one or more vents for gas to flow therethrough into/out from the enclosed space. In another embodiment, the measurement vessel can include a plate and a wall that are connected to each other to form the enclosed space, and the heater can be thermally coupled to the plate and/or the wall and configured to control the plate and/or the wall to reach the first temperature. In some embodiments, the measurement vessel can further include one or more vents formed in the plate and/or the wall for gas to flow therethrough into/out from the enclosed space. In various embodiments, the heater can be thermally coupled to the plate and configured to control the plate to reach the first temperature.

In an embodiment, the heater can be configured to send electrical charges through the pins to the wafer to heat up the wafer to the first temperature. In another embodiment, the bow measurement device can be configured to measure the wafer to identify the bow of the wafer when the enclosed space reaches the first temperature. In some embodiments, the bow measurement device can be configured to measure the wafer to identify the bow of the wafer when the enclosed space is at the second temperature.

In an embodiment, the wafer can be in contact with the measurement vessel when the pins are lowered to the second position. In another embodiment, the wafer can be in no contact with the measurement vessel when the pins are raised to the first position.

Aspects of the present disclosure also provide a method of operating a measurement device. The measurement device can include a measurement vessel providing an enclosed space for a wafer to be placed therein, and a plurality of pins provided in the measurement vessel for the wafer to rest thereon. The pins can be configured to be raised to a first position and lowered to a second position. The measurement device can also include a heater configured to control the enclosed space to reach a first temperature elevated from a second temperature, and a bow measurement device configured to measure the wafer to identify a bow of the wafer when the wafer rests on the pins and the pins are raised to the first position. For example, the method can include resting the wafer on the pins, the pins being lowered in the second position, raising the pins to the first position, using the heater to control the enclosed space to reach the first temperature, and measuring the wafer, using the bow measurement device, to identify a first bow of the wafer at the first temperature.

In an embodiment, the pins can be raised to the first position before the heater is used to control the enclosed space to reach the first temperature. In another embodiment, the pins can be raised to the first position after the heater is used to control the enclosed space to reach the first temperature.

In an embodiment, the method can further include measuring the wafer, using the bow measurement device, to identify a second bow of the wafer at the second temperature. In another embodiment, the wafer can be measured to identify the second bow of the wafer at the second temperature before the wafer is measured to identify the first bow of the wafer at the first temperature. In some embodiments, the method can further include lowering the pins to the second position after the wafer is measured to identify the second bow of the wafer, wherein the heater can be used to control the enclosed space to reach the first temperature when the pins are lowered to the second position. In various embodiments, the method can further include raising the pins to the first position after the heater is used to control the enclosed space to reach the first temperature. In yet another embodiment, the wafer can be in contact with the measurement vessel when the pins are lowered to the second position.

In another embodiment, the measurement vessel can include one or more vents for gas to flow therethrough into/out from the enclosed space, and the method can further include flowing the gas through the vents into/out from the enclosed space.

Note that this summary section does not specify every embodiment and/or incrementally novel aspect of the present disclosure or claimed invention. Instead, this summary only provides a preliminary discussion of different embodiments and corresponding points of novelty. For additional details and/or possible perspectives of the invention and embodiments, the reader is directed to the Detailed Description section and corresponding figures of the present disclosure as further discussed below.

### BRIEF DESCRIPTION OF THE DRAWINGS

Various embodiments of this disclosure that are proposed as examples will be described in detail with reference to the following figures, wherein like numerals reference like elements, and wherein:

FIGS. 1A-1C show first and second order bowing of a wafer;

FIG. 2 shows a bow measurement device that is used to measure a wafer to identify a bow of the wafer;

FIG. 3A is a schematic diagram of an exemplary measurement device according to some embodiments of the present disclosure;

FIG. 3B is a top view of the measurement device;

FIGS. 4A-4C illustrate an exemplary method of operating the measurement device of FIGS. 3A and 3B to measure a wafer in-situ at a predetermined (elevated) temperature to identify the bow of the wafer according to some embodiments of the present disclosure;

FIGS. 5A-5D illustrate another exemplary method of operating the measurement device of FIGS. 3A and 3B to measure a wafer in-situ at a predetermined (elevated) temperature to identify the bow of the wafer according to some embodiments of the present disclosure;

FIG. 6 is a schematic diagram of another exemplary measurement device according to some embodiments of the present disclosure; and

FIGS. 7A-7C illustrate yet another exemplary method of operating the measurement device of FIG. 6 to measure a wafer in-situ at a predetermined (elevated) temperature to identify the bow of the wafer according to some embodiments of the present disclosure.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed. Further, spatially relative terms, such as “top,” “bottom,” “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

The order of discussion of the different steps as described herein has been presented for clarity sake. In general, these steps can be performed in any suitable order. Additionally, although each of the different features, techniques, configurations, etc. herein may be discussed in different places of this disclosure, it is intended that each of the concepts can be executed independently of each other or in combination with each other. Accordingly, the present invention can be embodied and viewed in many different ways.

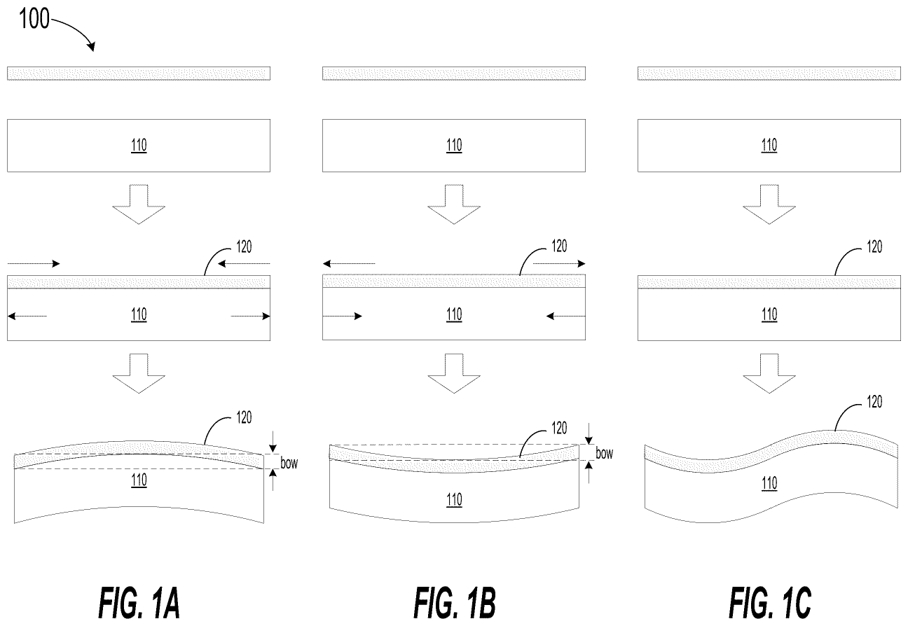

A functional semiconductor wafer can be comprised of the integration of 70+ individual layers that ultimately culminate in functional devices. Each level requires multiple processing steps including, but not limited to thin film deposition, lithography and etches to form the desired structures. For example, microfabrication of a semiconductor structure 100 begins with a flat substrate or wafer 110, as those illustrated in FIGS. 1A-1C. During microfabrication of the semiconductor structure 100, multiple processing steps are executed that can include depositing material on the wafer 110, removing material, implanting dopants, annealing, baking, and so forth. Different materials and structural formations 120 thus formed on the wafer 110 can induce non-uniform wafer stresses, which result in bowing of the wafer 110, which in turn affects dielectric film deposition uniformity and overlay and typically results in overlay errors of various magnitudes. For example, FIGS. 1A and 1B show how the different materials and structural formations 120 can either induce a compressive or tensile stress in the wafer 110, respectively, resulting in first order bow with bow measurements illustrating z-direction height (or z-height) deviations from a reference plane (not shown). As another example, FIG. 1C shows second order bow of the wafer 110 with two bow measurements identifying positive and negative z-height deviations, respectively.

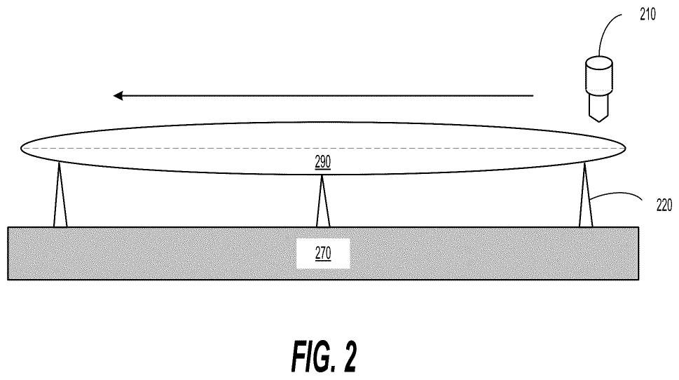

In an embodiment, a bow measurement device 210 (shown in FIG. 2) can use optical (e.g., using a one-dimensional (1D) scanning laser technique, or Xenon Lamp source technique with appropriate cooling assembly to prevent premature failure), capacitance-based, acoustic and Precise Focusing Objective mechanisms to measure the z-direction height deviations (i.e., the bow) across a surface of a wafer 290, e.g., the wafer 110. In an embodiment, the wafer 290 can be loaded onto a stage 270 with vacuum being off and then raised on a plurality of pins 220, e.g., three pins 220. The bow measurement device 210 can use objective focus (from the line-scan) to determine wafer-form W(r). The bow of the wafer 290 is the difference in height between the center and edge of the wafer 290, as shown in FIGS. 1A and 1B.

The bow of a wafer, e.g., the wafer 110, can be evaluated and measured under room temperature (RT) conditions. While adequate to access various materials contribution to the bow, the RT measurement fails to provide insight as to the stress of a wafer at an elevated temperature. For example, a deposition processing step is executed on a wafer in a deposition chamber at an elevated temperature (for example, a deposition temperature) that is higher than room temperature, and the stress of the wafer may be increased during the deposition processing step, which in turn could lead to significant wafer breaks and in severe cases substantial chamber damage.

Heat can be applied to a wafer in a manner outside of a measurement vessel, and then the heated wafer is transferred to the measurement vessel quickly to be measured. The inability to measure wafers in-situ can lead to significant cooling of the wafer upon transfer and inaccurate temperature correlation to the bow of the wafer.

Aspects of the present disclosure provide a measurement device that provides predictability of wafer breaks for customer to avoid significant tool downtime due to wafer breaks. For example, if the measurement device predicts wafer breaks during an elevated temperature, e.g., the deposition temperature, the wafer may be identified as being a “bad” wafer and no deposition processing step will be executed subsequently, a significant amount of time and resources thus saved. As another example, if the measurement device predicts no wafer breaks during an elevated temperature, e.g., the deposition temperature, the wafer may be identified as being a “good” wafer and the measurement data associated with the wafer can be fed into the recipe of the deposition processing step. The measurement data can be employed to adjust the recipe as needed, for example.

Memory customers who utilize high temperature deposition conditions for advanced patterning film (APF)/amorphous carbon layer (ACL) mask creation would highly benefit from this predictability capability. To elevate the temperature of a wafer and measure the bow of the wafer in-situ, a couple of hardware design modifications are proposed. In an embodiment, heating means (or a heater) can be provided that is configured to control the temperature, e.g., elevating or lowering the temperature, within a measurement vessel, in which a wafer can be placed. For example, addition of a heating plate and a heating wall will allow the wafer to reach elevated temperatures via conduction before pin up and by convection after pin up and during bow measurement. As another example, by electrifying the pins the wafer rests on, heat can be generated throughout the wafer assuming a semiconductor film is deposited on the backside surface of the wafer.

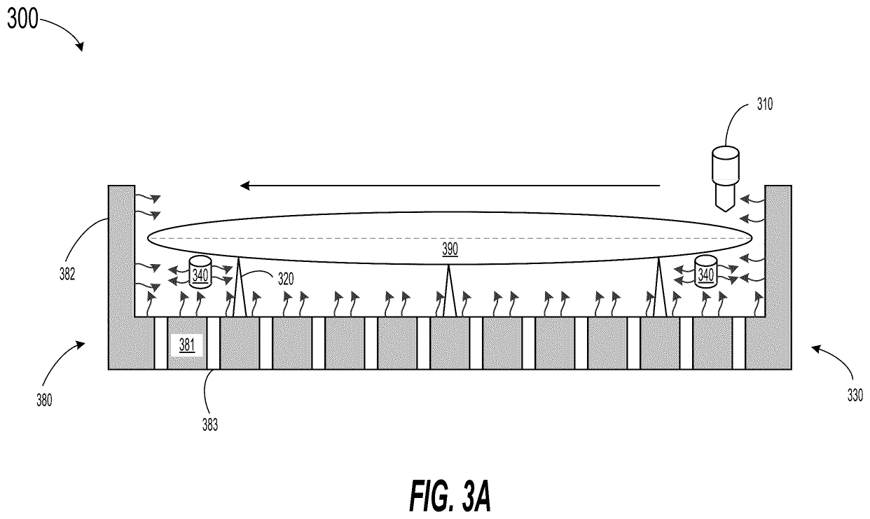

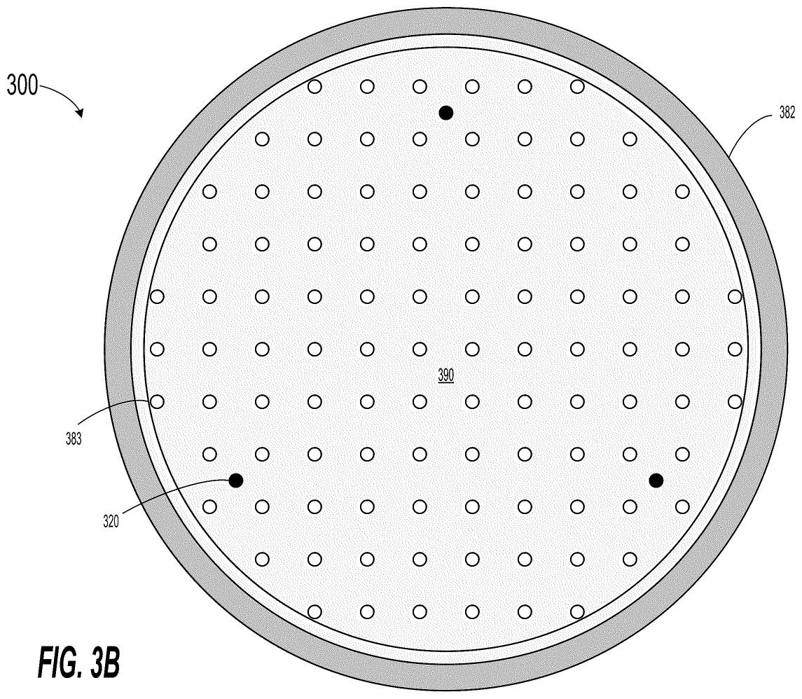

FIG. 3A is a schematic diagram of an exemplary measurement device 300 according to some embodiments of the present disclosure. FIG. 3B is a top view of the measurement device 300. The measurement device 300 can be used to measure a wafer, e.g., a wafer 390, in-situ at a predetermined temperature to identify the bow of the wafer 390. In an embodiment, the measurement device 300 can include a bow measurement device 310, a measurement vessel 380, a plurality of pins 320 and heating means (or a heater) 330.

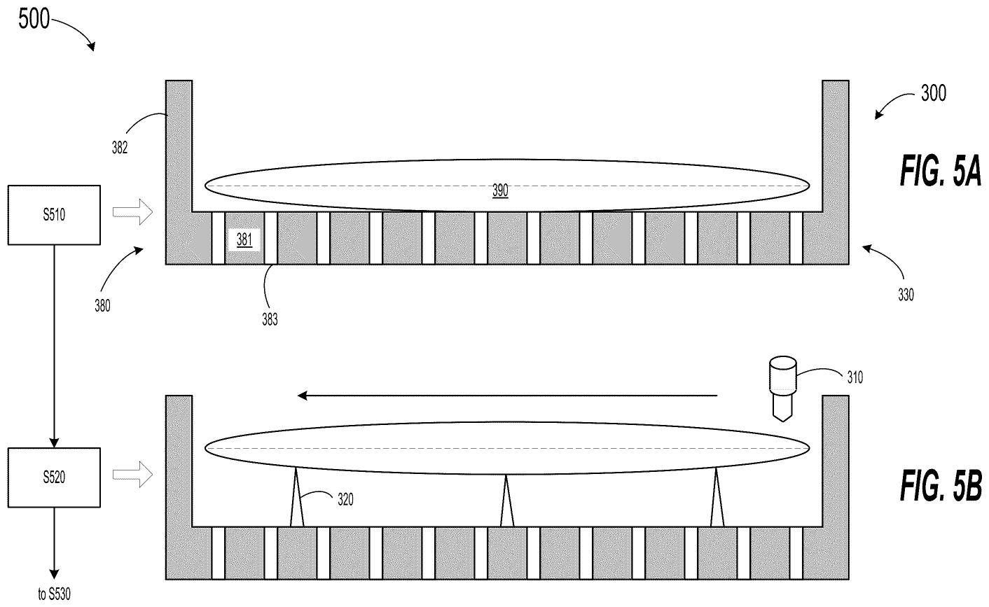

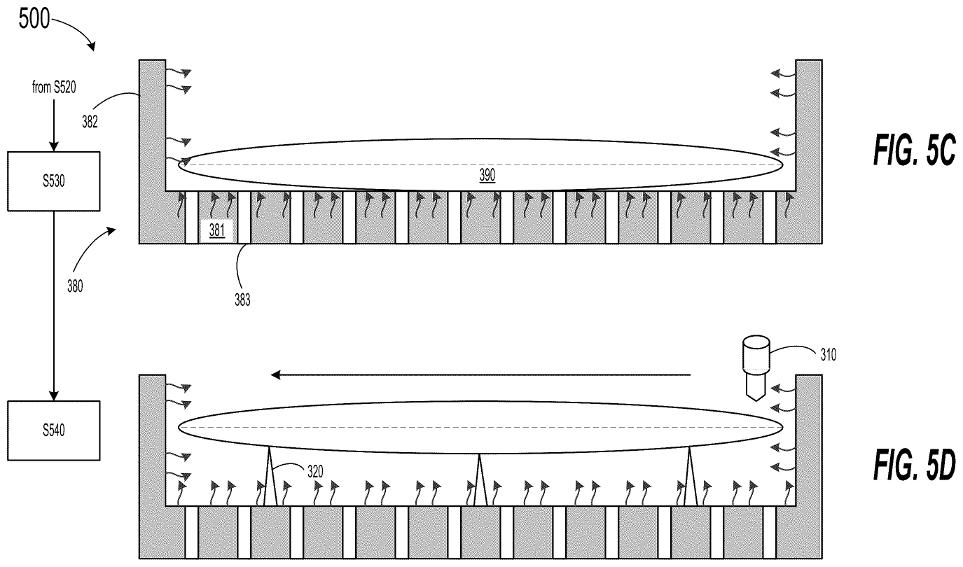

In an embodiment, a wafer, e.g., the wafer 390, can be placed within the measurement vessel 380 and rests on the pins 320 that are at an initial position. The pins 320 can be configured to be raised to move the wafer 390 upward to a measurement position such that the wafer 390 cannot be in contact with the heating means 330, as shown in FIG. 3A, and to be lowered to move the wafer 390 downward to the initial position such that the wafer 390 can be in contact with the heating means 330, as shown in FIG. 5C.

In an embodiment, the measurement vessel 380 can include a plate 381 and one or more walls 382 that are connected to each other to form an enclosed space, within which the wafer 390 can be placed. The plate 381 and/or the wall 382 can be made of thermally conductive materials, such as iron and copper. The heating means 330 can be thermally coupled to the plate 381 and/or the wall 382 and configured to control the plate 381 and/or the wall 382 to reach a predetermined temperature. Accordingly, the plate 381 and the wall 382 can act as a heating plate and a heating wall, respectively, to keep the enclosed space at the predetermined temperature. In an embodiment, the measurement vessel 380 can further include one or more vents 383 formed in the (heating) plate 381 and/or the (heating) wall 382 such that cooling gas, e.g., nitrogen (N₂) gas, can flow via the vents 383 into and out from the enclosed space to cool off the wafer 390. In some embodiments, the heating means 330 can further include one or more heating devices 340, e.g., infrared-heating lamps, that are located within the measurement vessel 380 to act along, or together with the (heating) plate 381 and/or the (heating) wall 382, to keep the enclosed space at the predetermined temperature.

In an embodiment, the bow measurement device 310 can use optical (e.g., using a one-dimensional (indicated by the arrow) scanning laser technique), capacitance-based, acoustic and other mechanisms to measure the z-direction height deviations (i.e., the bow) across a surface of the wafer 390.

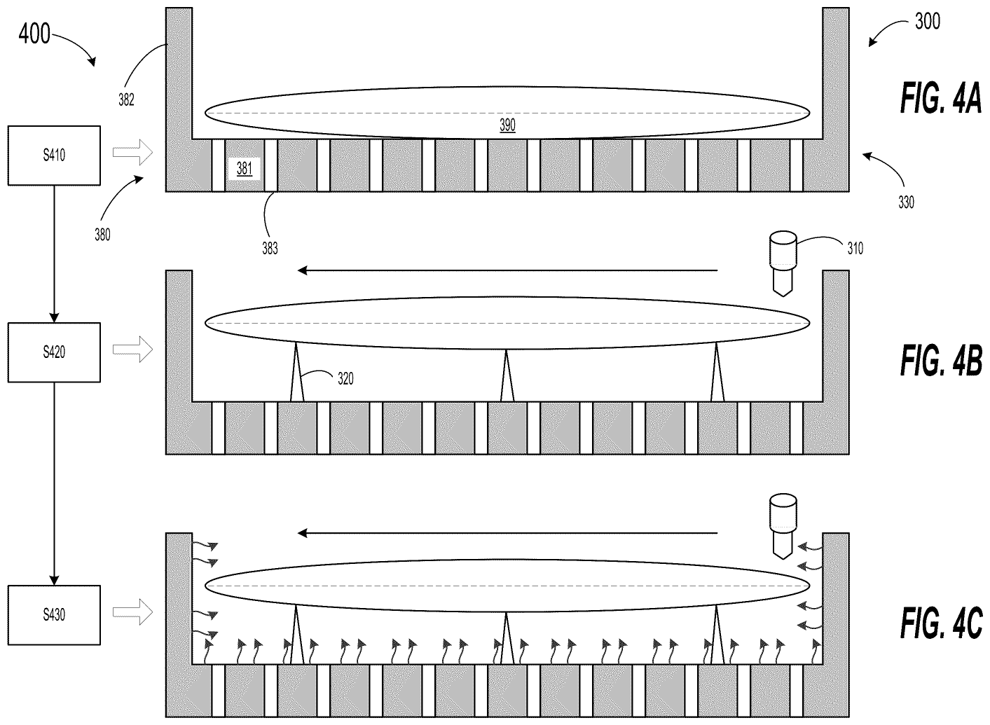

FIGS. 4A-4C illustrate an exemplary method 400 of operating a measurement device, e.g., the measurement device 300, to measure a wafer, e.g., the wafer 390, in-situ at a predetermined (elevated) temperature to identify the bow of the wafer 390 according to some embodiments of the present disclosure. In various embodiments, some of the steps of the method 400 shown can be performed concurrently or in a different order than shown, can be substituted by other method steps, or can be omitted. Additional method steps can also be performed as desired. The method 400 can start at step S410.

At step S410, the pins 320 of the measurement device 300 can be lowered to the initial position, and the wafer 390 can be placed within the measurement vessel 380 of the measurement device 300 and rest on the lowered pins 320, as shown in FIG. 4A. The method 400 can then proceed to step S420.

At step S420, the pins 320 can be raised and move the wafer 390 to the measurement position, and the bow measurement device 310 can measure the wafer 390 (1D or 2D scan, for example) at room temperature (RT), as shown in FIG. 4B. For example, the bow measurement device 310 can take 1D scan by moving along the diameter of the wafer 390 and measure the wafer 390 to identify the 1D bow of the wafer 390. The method 400 can then proceed to step S430.

At step S430, the heating means 330 (or the (heating) plate 381 and/or the (heating) wall 382 (and/or the heating device 340) can be activated, allowing the measurement vessel 380 to reach a predetermined (elevated) temperature, e.g., a deposition temperature, and the bow measurement device 310 can measure the wafer 390 (1D or 2D scan, for example) to identify the bow of the wafer 390 at the predetermined elevated temperature, as shown in FIG. 4C. As the wafer 390 rests on the pins 320 that are raised and is thus in no contact with the (heating) plate 381 and the (heating) wall 382, the wafer 390 can reach the predetermined elevated temperature via convection formed by the (heating) plate 381 and/or the (heating) wall 382 within the measurement vessel 380 during the bow measurement process. After the bow of the wafer 390 is identified, cooling gas, e.g., nitrogen gas, can flow via the vents 383 into and out from the enclosed space of the measurement vessel 380 to cool off the wafer 390. In an embodiment, the cooling gas can flow under the wafer 390 for quick temperature quenching.

FIGS. 5A-5D illustrate an exemplary method 500 of operating a measurement device, e.g., the measurement device 300, to measure a wafer, e.g., the wafer 390, in-situ at a predetermined (elevated) temperature to identify the bow of the wafer 390 according to some embodiments of the present disclosure. In various embodiments, some of the steps of the method 500 shown can be performed concurrently or in a different order than shown, can be substituted by other method steps, or can be omitted. Additional method steps can also be performed as desired. The method 500 can start at step S510.

At step S510, which is similar to step S410 of the method 400, the pins 320 of the measurement device 300 can be lowered to the initial position, and the wafer 390 can be placed within the measurement vessel 380 of the measurement device 300 and rest on the lowered pins 320, as shown in FIG. 5A. The method 500 can then proceed to step S520.

At step S520, which is similar to step S420 of the method 400, the pins 320 can be raised and move the wafer 390 upward to the measurement position, and the bow measurement device 310 can measure the wafer 390 at room temperature (RT), as shown in FIG. 5B. The method 500 can then proceed to step S530.

At step S530, the heating means 330 (or the (heating) plate 381 and/or the (heating) wall 382) can be activated, allowing the measurement vessel 380 to reach a predetermined (elevated) temperature, e.g., a deposition temperature, and the pins 320 can be lowered to the initial position such that the wafer 390 can be in contact with the (heating) plate 381. As the wafer 390 is in contact with the (heating) plate 381 and within the measurement vessel 380, the wafer 390 can reach the predetermined temperature, via thermal conduction formed by the (heating) plate 381 and convection formed by the (heating) plate 381 and the (heating) wall 382 within the measurement vessel 380, more quickly than the wafer 390 does at step S430 of the method 400, at which the wafer 390 is heated up via the convection only. In an embodiment, the pins 320 can be lowered to the initial position such that the wafer 390 can be in contact with the (heating) plate 381 first, and then the heating means 330 (or the (heating) plate 381 and/or the (heating) wall 382) can be activated, allowing the measurement vessel 380, and the wafer 390 as well, to reach the predetermined (elevated) temperature. The method 500 can then proceed to step S540.

At step S540, the pins 320 can be raised again and move the wafer 390 upward to reach the measurement position, and the bow measurement device 310 can measure the wafer 390 to identify the bow of the wafer 390 at the predetermined elevated temperature. At step S540, while the wafer 390 is raised by the pins 320 and is in no contact with the (heating) plate 381, the convection formed by the (heating) plate 381 and the (heating) wall 382 within the measurement vessel 380 can still maintain the wafer 390 at the predetermined (elevated) temperature.

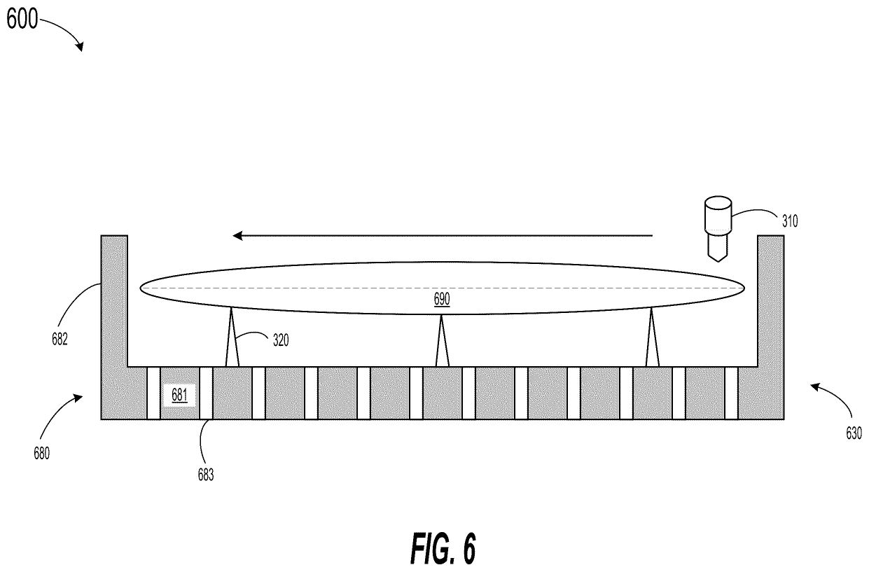

FIG. 6 is a schematic diagram of an exemplary measurement device 600 according to some embodiments of the present disclosure. The measurement device 600 can be used to measure a wafer, e.g., a wafer 690, in-situ at a predetermined (elevated) temperature to identify the bow of the wafer 690. In an embodiment, the measurement device 600 can include the bow measurement device 310, a measurement vessel 680, the pins 320 and heating means (or a heater) 630.

In an embodiment, a wafer, e.g., the wafer 690, can be placed within the measurement vessel 680 and rests on the pins 320.

In an embodiment, the measurement vessel 680 can include a plate 681 and one or more walls 682 that are connected to each other to form an enclosed space, within which the wafer 690 can be placed. In another embodiment, the measurement vessel 680 can further include one or more vents 683 formed in the plate 681 and/or the wall 682 such that cooling gas, e.g., nitrogen (N₂) gas, can flow via the vents 683 into and out from the enclosed space to cool off the wafer 690.

In an embodiment, the heating means 630 can be configured to electrify the wafer 690, for example, by sending electrical charges through the pins 320 to the wafer 690, to heat up the wafer 690 to a predetermined (elevated) temperature. For example, a semiconductor layer, e.g., doped with Si, can be formed on a backside surface of the wafer 690, and the heating means 630 can send electrical charges through the pins 320 to the backside surface of the wafer 690 when the wafer 690 is resting on the pins 320 to elevate the temperature of the wafer 690 to a predetermined temperature, e.g., the deposition temperature, whether or not the pins 320 are raised to the measurement position or lowered to the initial position.

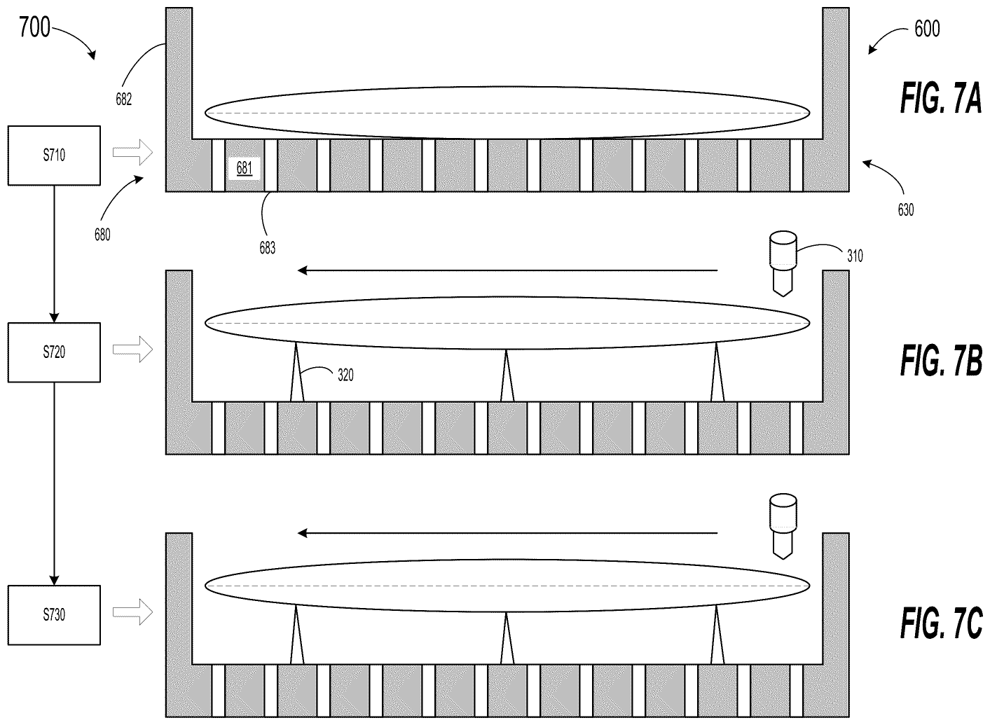

FIGS. 7A-7C illustrate an exemplary method 700 of operating a measurement device, e.g., the measurement device 600, to measure a wafer, e.g., the wafer 690, in-situ at a predetermined (elevated) temperature to identify the bow of the wafer 690 according to some embodiments of the present disclosure. In various embodiments, some of the steps of the method 700 shown can be performed concurrently or in a different order than shown, can be substituted by other method steps, or can be omitted. Additional method steps can also be performed as desired. The method 700 can start at step S710.

At step S710, which is similar to step S410 of the method 400, the pins 320 of the measurement device 600 can be lowered to the initial position, and the wafer 690 can be placed within the measurement vessel 680 of the measurement device 600 and rest on the lowered pins 320, as shown in FIG. 7A. The method 700 can then proceed to step S720.

At step S720, which is similar to step S420 of the method 400, the pins 320 can be raised and move the wafer 690 upward to the measurement position, and the bow measurement device 310 can measure the wafer 690 (1D or 2D scan, for example) at room temperature (RT), as shown in FIG. 7B. For example, the bow measurement device 310 can take 1D scan by moving along the diameter of the wafer 690 and measure the wafer 690 to identify the 1D bow of the wafer 690. The method 700 can then proceed to step S730.

At step S730, the heating means 630 can send electrical charges via the pins 320 to (the backside surface of) the wafer 690 to heat up the wafer 690 to the predetermined elevated temperature, and the bow measurement device 310 can measure the wafer 690 (1D or 2D scan, for example) to identify the bow of the wafer 690 at the predetermined elevated temperature, as shown in FIG. 7C. After the bow of the wafer 690 is identified, cooling gas, e.g., nitrogen gas, can flow via the vents 683 into and out from the enclosed space of the measurement vessel 680 to cool off the wafer 690.

In an embodiment, the plate 681 and/or the wall 682 of the measurement vessel 680 can be made of thermally conductive materials, such as iron and copper, and the heating means 630 can be thermally coupled to the plate 681 and/or the wall 682 and further configured to control the plate 681 and/or the wall 682 to reach the predetermined temperature. Accordingly, the plate 681 and the wall 682 can act as a heating plate and a heating wall, respectively, to keep the enclosed space at the predetermined temperature.

In another embodiment, the heating means 330 of the measurement device 300 can be further configured to send electrical charges via the pins 320 to the wafer 390 to heat up the wafer 390 to the predetermined temperature.

In some embodiments, the heating means 330 and 630 can be configured to emit microwaves and/or radiation light to elevate the temperature of the enclosed space of the measurement vessels 380 and 680 and/or the wafers 390 and 690 to the predetermined temperature.

In various embodiments, the measurement devices 300 and 600 can further include a temperature sensor, e.g., a thermometer, that can sense the temperatures of the enclosed spaces of the measurement devices 380 and 680 and the wafers 390 and 690, and, accordingly, the heating means 330 and 630 can be controlled to be activated or deactivated by determining whether the temperature that the thermometer senses reaches the predetermined (elevated) temperature.

In the preceding description, specific details have been set forth, such as a particular geometry of a processing system and descriptions of various components and processes used therein. It should be understood, however, that techniques herein may be practiced in other embodiments that depart from these specific details, and that such details are for purposes of explanation and not limitation. Embodiments disclosed herein have been described with reference to the accompanying drawings. Similarly, for purposes of explanation, specific numbers, materials, and configurations have been set forth in order to provide a thorough understanding. Nevertheless, embodiments may be practiced without such specific details. Components having substantially the same functional constructions are denoted by like reference characters, and thus any redundant descriptions may be omitted.

Various techniques have been described as multiple discrete operations to assist in understanding the various embodiments. The order of description should not be construed as to imply that these operations are necessarily order dependent. Indeed, these operations need not be performed in the order of presentation. Operations described may be performed in a different order than the described embodiment. Various additional operations may be performed and/or described operations may be omitted in additional embodiments.

“Substrate” or “target substrate” as used herein generically refers to an object being processed in accordance with the invention. The substrate may include any material portion or structure of a device, particularly a semiconductor or other electronics device, and may, for example, be a base substrate structure, such as a semiconductor wafer, reticle, or a dielectric layer on or overlying a base substrate structure such as a thin film. Thus, substrate is not limited to any particular base structure, underlying dielectric layer or overlying dielectric layer, patterned or un-patterned, but rather, is contemplated to include any such dielectric layer or base structure, and any combination of dielectric layers and/or base structures. The description may reference particular types of substrates, but this is for illustrative purposes only.

Those skilled in the art will also understand that there can be many variations made to the operations of the techniques explained above while still achieving the same objectives of the invention. Such variations are intended to be covered by the scope of this disclosure. As such, the foregoing descriptions of embodiments of the invention are not intended to be limiting. Rather, any limitations to embodiments of the invention are presented in the following claims.

What is claimed is:

1. A measurement device, comprising:
a measurement vessel providing an enclosed space for a wafer to be placed therein;
a plurality of pins provided in the measurement vessel for the wafer to rest thereon, the pins configured to be raised to a first position and lowered to a second position;
a heater configured to control the enclosed space to reach a first temperature elevated from a second temperature; and
a bow measurement device configured to measure the wafer to identify a bow of the wafer when the wafer rests on the pins and the pins are raised to the first position.

2. The measurement device of claim 1, the measurement vessel further includes one or more vents for gas to flow therethrough into/out from the enclosed space.

3. The measurement device of claim 1, wherein the measurement vessel includes a plate and a wall that are connected to each other to form the enclosed space, and the heater is thermally coupled to the plate and/or the wall and configured to control the plate and/or the wall to reach the first temperature.

4. The measurement device of claim 3, wherein the measurement vessel further includes one or more vents formed in the plate and/or the wall for gas to flow therethrough into/out from the enclosed space.

5. The measurement device of claim 3, wherein the heater is thermally coupled to the plate and configured to control the plate to reach the first temperature.

6. The measurement device of claim 5, wherein the measurement vessel further includes one or more vents formed in the plate for gas to flow therethrough into/out from the enclosed space.

7. The measurement device of claim 1, wherein the heater is configured to send electrical charges through the pins to the wafer to heat up the wafer to the first temperature.

8. The measurement device of claim 1, wherein the bow measurement device is configured to measure the wafer to identify the bow of the wafer when the enclosed space reaches the first temperature.

9. The measurement device of claim 1, wherein the bow measurement device is configured to measure the wafer to identify the bow of the wafer when the enclosed space is at the second temperature.

10. The measurement device of claim 1, wherein the wafer is in contact with the measurement vessel when the pins are lowered to the second position.

11. The measurement device of claim 1, wherein the wafer is in no contact with the measurement vessel when the pins are raised to the first position.

12. A method of operating a measurement device, the measurement device comprising:
a measurement vessel providing an enclosed space for a wafer to be placed therein;
a plurality of pins provided in the measurement vessel for the wafer to rest thereon, the pins configured to be raised to a first position and lowered to a second position;
a heater configured to control the enclosed space to reach a first temperature elevated from a second temperature; and
a bow measurement device configured to measure the wafer to identify a bow of the wafer when the wafer rests on the pins and the pins are raised to the first position,
the method comprising:
resting the wafer on the pins, the pins being lowered in the second position;
raising the pins to the first position;
using the heater to control the enclosed space to reach the first temperature; and
measuring the wafer, using the bow measurement device, to identify a first bow of the wafer at the first temperature.

13. The method of claim 12, wherein the pins are raised to the first position before the heater is used to control the enclosed space to reach the first temperature.

14. The method of claim 12, wherein the pins are raised to the first position after the heater is used to control the enclosed space to reach the first temperature.

15. The method of claim 12, further comprising:
measuring the wafer, using the bow measurement device, to identify a second bow of the wafer at the second temperature.

16. The method of claim 15, wherein the wafer is measured to identify the second bow of the wafer at the second temperature before the wafer is measured to identify the first bow of the wafer at the first temperature.

17. The method of claim 16, further comprising:
lowering the pins to the second position after the wafer is measured to identify the second bow of the wafer,
wherein the heater is used to control the enclosed space to reach the first temperature when the pins are lowered to the second position.

18. The method of claim 17, further comprising:
raising the pins to the first position after the heater is used to control the enclosed space to reach the first temperature.

19. The method of claim 17, wherein the wafer is in contact with the measurement vessel when the pins are lowered to the second position.

20. The method of claim 12, wherein the measurement vessel includes one or more vents for gas to flow therethrough into/out from the enclosed space, and the method further includes flowing the gas through the vents into/out from the enclosed space.
